SEQUENCE LISTING (12 sequences)

SEQ ID NO: 1 - DNA, 1131 bp, Moorella thermoacetica
  CDS (1)..(1131)
atgggcttctatgatgtgtataaacagtatgaaggctttgactttgaaggctttttccaatcgcgcaccccggatgacgttcgtaaagcgctggccaaagaacatctggaagtgaccgattatctgaccctgctgtctccggccgcgggtaactttctggaagaaatggcccaaaaagcacaccgtattaccctgcgcaatttcggtcgtgtcatctttctgttcacgccgctgtatctgtcagattactgcgtgaaccagtgcgcgtattgttcgtttaacgctcgcaataaattcgcgcgtaccaaactgacgctggaacaagttgaagaagaagcgcgcgctattgcgcagaccggcatgaaagatattctgatcctgacgggtgaaagtcgtcaacataatccggtttcctatatcaaagactgtgtgggcgttctgaaaaaatacttctgcagtatttgtatcgaagtctatccgctggaagaagaagaataccgcgaactggtcgcagctggcgtggatggtctgaccatgttccaggaagtttatgacccgggcgtctatgcccgctaccataacggtccgaagaaaaactatcactaccgtctggatgcgccggaacgtagctgtcgtgccggtatgcgtaccgttggtgttggtgcactgctgggtctggcagactggcgtaaagaagcgtttttcaccggcctgcatgcagattatctgcagcaaaaattttgggacgttcaagtcagtatttccctgccgcgttttcgtccgagcatcggcggtttccaaccggattacccggtggatgacaaatcttttgttcagattctgctggcccaccgtctgttcctgccgcgtgtgggtattaccatctcaacgcgcgaatcgccggaatttcgtgataacattctgccgctgggcgttaccaaaatcagcgccggtagctctgtgacggttggcggttatgcgcgtccggatggtatggccccgcagttcgaaattagcgacccgcgttctgtggcggaaattaaacagatgctgatccaaaaaggttaccagccggtgtttgaagactggcagcaatgggactcactggaaaaacaactgtataacttctaa

SEQ ID NO: 2 - protein, 376 aa, Moorella thermoacetica
MGFYDVYKQYEGFDFEGFFQSRTPDDVRKALAKEHLEVTDYLTLLSPAAGNFLEEMAQKAHRITLRNFGRVIFLFTPLYLSDYCVNQCAYCSFNARNKFARTKLTLEQVEEEARAIAQTGMKDILILTGESRQHNPVSYIKDCVGVLKKYFCSICIEVYPLEEEEYRELVAAGVDGLTMFQEVYDPGVYARYHNGPKKNYHYRLDAPERSCRAGMRTVGVGALLGLADWRKEAFFTGLHADYLQQKFWDVQVSISLPRFRPSIGGFQPDYPVDDKSFVQILLAHRLFLPRVGITISTRESPEFRDNILPLGVTKISAGSSVTVGGYARPDGMAPQFEISDPRSVAEIKQMLIQKGYQPVFEDWQQWDSLEKQLYNF

SEQ ID NO: 3 - DNA, 1068 bp, Chlorobium tepidum
  CDS (1)..(1068)
atgattgcgctgcccgcatggctgaccgacgagcggctgtcggaagatatcgaaccgctgttgcgacaaacggataacgagtcgctcgaacggcttgccgccgaagcgcaggcagtgacactgcgccgtttcgggcgcgtcatttcgctctatacgccgctctacctctccaacttctgctcgagcggttgcgtctattgcggcttcgcttcggacagacgttcgccgcgccgcaagctggatactgacgaaatcgaaaaggagctgctcgcaatgaaggctctcggcgtcagcgacgttttgctgctcaccggcgagcgcaccaactcggtgggattcgactatctgcgtcgcgccgtggatatcgccgcccgccacatgccgcgcgtagccgtcgaggcgtttccgatgagcgtcgcagagtatcgcggcctggccgaatgtgggtgcaccggcctgacgatttaccaggaaacctacgatccggatcattaccgcgagctgcaccgctgggggccgaagcaggatttcctcgaacggctcgaaacgccggaacgcgccatcaccggcggcatccggagcgtcggcatcggcgcactgctcggcctgtcggagccggtcggcgaagcgctcgccgtgttgcgccacgcgcggtatctgtgcaaaacgtactggaaagcaggcgtcacggtctcctttccccgcatccgcccgcaggagggcggctttcagcccagcttcacggtctcggatcgcttcctcgcacgaatgatcttcgccttccgcatcggaatgccggatgtcgatctggtgctctcgacgcgagagagttcgaattttcgggacggcatggctggcctcggcatcacccgcatgagcatcgccagccgcaccaccgttggcggctacgtcgaaaaggagacggctggagccagccagttcgaggtgagcgacaaccgaagcgtcgaagcgttttgcgccgcattgcgcgcaaaagatctggaaccagtgttcaaaaactgggacgcggcctacaacaaccccctgcccgcagaggagtgcacgtga

SEQ ID NO: 4 - protein, 355 aa, Chlorobium tepidum
MIALPAWLTDERLSEDIEPLLRQTDNESLERLAAEAQAVTLRRFGRVISLYTPLYLSNFCSSGCVYCGFASDRRSPRRKLDTDEIEKELLAMKALGVSDVLLLTGERTNSVGFDYLRRAVDIAARHMPRVAVEAFPMSVAEYRGLAECGCTGLTIYQETYDPDHYRELHRWGPKQDFLERLETPERAITGGIRSVGIGALLGLSEPVGEALAVLRHARYLCKTYWKAGVTVSFPRIRPQEGGFQPSFTVSDRFLARMIFAFRIGMPDVDLVLSTRESSNFRDGMAGLGITRMSIASRTTVGGYVEKETAGASQFEVSDNRSVEAFCAALRAKDLEPVFKNWDAAYNNPLPAEECT

SEQ ID NO: 5 - DNA, 1122 bp, Carboxythermus hydrogenoformans
  CDS (1)..(1122)
atgctggactacctgcaaaaatgctttgaactgtactttcgttatgaacaatacgtgccgtcgtttagcgaagctctggacatcctggcgaaagattatctggacaaaattgatctggttaaactgctgaacgtcgaagatgaagaaatcatcaaattcatggcgaaaaaagccaaacgtatcaccgaactgaatttcggcaaagtgattctgctgtatgcaccgctgtacatcgctaacttttgcgaaaatggctgcgtttattgtggtttctctaaactgcgtaaatacccgcgcgaaaaactgagtctggaacagatggaagaagaaatgcagcaaattaaatcagaaggcattgactcgatcctgctgctgaccggtgaagatcgtaaaaacagcccgttcgcatacatcaaaaacgcatgtaaactggctacgaaatacttcagcgaagtctctatcgaagtgtatccgctgagtaaagaagaatacgaagaactggctcgcattggcgttatcggtaccacgatttatcaggaaacctacatcaaaaaagactacgaaaaactgcacctgtttggcccgaaaaaagattatgaatttcgtctgtacacgccggaacgcgcactgaaagctggttttaaagcggcctcaattggcccgctgctgggtctgtcgctgccgaaactggacgtgtatagcgcgatcctgcatgccgattatctgatgaaaaaatacccgcaagcggaaattgccatctcctttccgcgtctgcgcgcagctaacaccggcttcaaagcaaaacacgtggtttctgataaagaatttatcaaattcctgctggtcacccgtatctatctgccgcgcattggtatcaatctgagtacccgcgaacgcccgtccctgcgtgatgccctgctggatatttgcatcaccaaaatgagtgccggctccaaaaccacggtgggcggttactttagcaaaaaagaagactctcagggtcaattcgaagtggaagatcgtcgcatggttgccgaaattatcgaagtcattcgcaaaaaaggtctgcgcccggaatttaccaactggattcgcggtgttcgcccgtatgaactgctgtga

SEQ ID NO: 6 - protein, 373 aa, Carboxythermus hydrogenoformans
MLDYLQKCFELYFRYEQYVPSFSEALDILAKDYLDKIDLVKLLNVEDEEIIKFMAKKAKRITELNFGKVILLYAPLYIANFCENGCVYCGFSKLRKYPREKLSLEQMEEEMQQIKSEGIDSILLLTGEDRKNSPFAYIKNACKLATKYFSEVSIEVYPLSKEEYEELARIGVIGTTIYQETYIKKDYEKLHLFGPKKDYEFRLYTPERALKAGFKAASIGPLLGLSLPKLDVYSAILHADYLMKKYPQAEIAISFPRLRAANTGFKAKHVVSDKEFIKFLLVTRIYLPRIGINLSTRERPSLRDALLDICITKMSAGSKTTVGGYFSKKEDSQGQFEVEDRRMVAEIIEVIRKKGLRPEFTNWIRGVRPYELL

SEQ ID NO: 7 - DNA, 1107 bp, Clostridium acetobutylicum
  CDS (1)..(1107)
atgagcttctacaaaaaactgcaacaatacaaagacttcgatttcgacgacttcttcagcaaagtgacggcacgcgacattgaaaaaatcctgtgcaaagatattctgcatgaaatggactttctgaaactgctgagcccggcggccgaaaaatacctggaacacatggcacagaaagctcgtgaactgtctctgaaaaacttcggcaaaaccgtggttctgtatacgccgatttacatcgcaaactattgtgtgaatggctgcgcttactgtggttacaacgttaaaaacaaaattaaacgcaaacagctgacgatggaagaaatcgaagaagaagcgcgtgctatttatagctctggcatgcgcaacattatcctgctgaccggtgaaagcaaagtgcaaacgccggtttcttacatcaaagatgcgatcaaactgctgaaaaaatacttcagttccatttgcatcgaaatttacccgctggaagttaatgaatatcgtgaactggtcgaagcgggcgccgatagcctgaccatctaccaagaaacgtacaacgaagaaaaatactcaaaagtccacctgtcgggtccgaaacgtaattttaaattccgcctggacgcgccggaacgtgtttgtgaagccggcatccattcaattggcaccggtgccctgctgggtctgtacaaatggcgctcggaagcatttttcacgggcctgcacgctagttatatccaggaaaaatttccgtccgtggaaatctcaatgagcgccccgcgtattcgcccgcatgcaggtagcttcgatgacatctacgaagttaacgataaaaacatcgtccaagtgattctggcctataaaatgtttctgccgcgtgcaggcaccaacatcaccacgcgtgaaccgaaagaatttcgcgataaactgatcccgattggcgtgaccaaaatgagtgcgggtgtctccacggaagtgggcggtcatggttgcaaagacaaaggcgaaggccagttcgataccaatgacaaacgcagcgtttctgaagtctataatcgtatcaaagaactgggctacaacccggtgttcaaagacttcgaaaacgcactgtga

SEQ ID NO: 8 - protein, 368 aa, Clostridium acetobutylicum
MSFYKKLQQYKDFDFDDFFSKVTARDIEKILCKDILHEMDFLKLLSPAAEKYLEHMAQKARELSLKNFGKTVVLYTPIYIANYCVNGCAYCGYNVKNKIKRKQLTMEEIEEEARAIYSSGMRNIILLTGESKVQTPVSYIKDAIKLLKKYFSSICIEIYPLEVNEYRELVEAGADSLTIYQETYNEEKYSKVHLSGPKRNFKFRLDAPERVCEAGIHSIGTGALLGLYKWRSEAFFTGLHASYIQEKFPSVEISMSAPRIRPHAGSFDDIYEVNDKNIVQVILAYKMFLPRAGTNITTREPKEFRDKLIPIGVTKMSAGVSTEVGGHGCKDKGEGQFDTNDKRSVSEVYNRIKELGYNPVFKDFENAL

SEQ ID NO: 9 - DNA, 1134 bp, Escherichia coli
  CDS (1)..(1134)
atgaaaaccttcagcgatcgctggcgacaactggactgggacgacatccgcctgcgtatcaacggcaaaacggctgctgacgtagagcgggcgctaaatgcctcgcaactcacccgcgacgacatgatggcgctgttatcgcctgccgccagtggctatctggaacaactggcccaacgggcgcagcgtctgacccgtcagcgatttggcaacacagttagtttctacgtcccgctttatctttccaatctttgcgctaacgactgcacgtactgtggattttccatgagtaatcgcatcaagcgcaaaacgctggatgaagcggatattgccagggaaagtgccgctatacgggagatgggctttgaacatctgctgttagtcactggtgaacatcaggcgaaagtggggatggattactttcgtcgtcatctccctgcccttcgtgaacagttctcttcactacagatggaagtgcaaccgctggcggagacggaatacgccgagttaaagcaacttggtctggatggcgtgatggtttatcaggagacatatcacgaggcgacttatgcccgccatcatctgaaaggcaaaaaacaggacttcttctggcggctggaaacgccggatcggctggggcgtgcggggattgataagataggcctcggcgcgctaattggcctttccgacaactggcgcgttgacagctatatggttgccgaacatttgctatggctgcaacagcattactggcaaagccgttactctgtctcctttccgcgcctgcgcccgtgtactggcggcattgagcctgcgtcgattatggatgaacgccagttagtgcaaaccatctgcgccttccgactgcttgcaccggagattgaactgtcactctccacgcgggaatcaccgtggtttcgcgatcgcgttattccgctggcgatcaataacgtcagcgccttctcgaaaacgcagccaggtggctatgccgataatcaccccgagttggaacagttctcaccgcacgacgatcgcagaccggaagcggttgctgccgcgttaaccgctcagggtttgcagccggtatggaaagactgggacagctatctgggacgcgcctcgcaaagactatga

SEQ ID NO: 10 - protein, 377 aa, Escherichia coli
MKTFSDRWRQLDWDDIRLRINGKTAADVERALNASQLTRDDMMALLSPAASGYLEQLAQRAQRLTRQRFGNTVSFYVPLYLSNLCANDCTYCGFSMSNRIKRKTLDEADIARESAAIREMGFEHLLLVTGEHQAKVGMDYFRRHLPALREQFSSLQMEVQPLAETEYAELKQLGLDGVMVYQETYHEATYARHHLKGKKQDFFWRLETPDRLGRAGIDKIGLGALIGLSDNWRVDSYMVAEHLLWLQQHYWQSRYSVSFPRLRPCTGGIEPASIMDERQLVQTICAFRLLAPEIELSLSTRESPWFRDRVIPLAINNVSAFSKTQPGGYADNHPELEQFSPHDDRRPEAVAAALTAQGLQPVWKDWDSYLGRASQRL

SEQ ID NO: 11 - DNA, 37 bp, artificial sequence
  primer
ggtaatccatatgattgcgctgcccgcatggctgacc

SEQ ID NO: 12 - DNA, 36 bp, artificial sequence
  primer
gggaattcttatcacgtgcactcctctgcgggcagg

(19) United States
(12) Patent Application Publication
(10) Pub. No.: US 2016/0376579 A1
(43) Pub. Date: Dec. 29, 2016

(54) NEW ENZYMES AND METHOD FOR PREPARING 4-HYDROXYL BENZYL ALCOHOL AND DERIVATIVES THEREOF

(71) Applicants: COMMISSARIAT A L'ENERGIE ATOMIQUE ET AUX ENERGIES ALTERNATIVES, Paris (FR); INTITUT NATIONAL DE LA RECHERCHE AGRONOMIQUE (INRA), Paris (FR)

(72) Inventors: OLIVIER BERTEAU, JOUY EN JOSAS (FR); YVAIN NICOLET, VIZILLE (FR); JUAN FONTECILLA-CAMPS, LES ADRETS (FR); ALHOSNA BENJDIA, LIMEIL BREVANNES (FR); ALAIN GUILLOT, FORGES LES BAINS (FR)

(21) Appl. No.: 15/039,865

(22) PCT Filed: Dec. 18, 2014
(86) PCT No.: PCT/EP2014/078405

§ 371 Date: May 27, 2016

(30) Foreign Application Priority Data
Dec. 18, 2013 (EP) 13306752.0

(51) Int. Cl. C12N 9/88; C12N 15/70; C12P 7/22

(52) U.S. Cl. CPC C12N 9/88; C12P 7/22; C12Y 403/01023; C12N 15/70

(57) ABSTRACT

The present invention relates to a new enzyme able to produce 4-hydroxybenzyl alcohol from the amino acid tyrosine and the use thereof for producing 4-hydroxybenzyl alcohol.

### FIELD OF THE INVENTION

The present invention relates to a new enzyme and its use in methods for preparing compounds of interest.

### BACKGROUND OF THE INVENTION

Aromatic compounds, including hydroxyl benzyl alcohols (HBA's), which are intermediates in the manufacturing of dyes, pharmaceutical products, additives, and polymers, are key elements in the chemical industry. Furthermore, HBA's have interesting biological functions and properties, exhibiting notably an excellent neuroprotective effect and are effective free radical scavengers.

p-Hydroxybenzyl alcohol (4-HBA) and its derivatives are important starting materials for the synthesis of useful organic compounds including pharmaceutical compounds such as the cardioselective β1-adrenergic blocking agent bisoprolol (WO/2007/069266/Arcelor Ltd); vanillin (Rhodia), various chemicals such as p-hydroxybenzylaldehyde, 4,4′-dihydroxydiphenylmethane and polymers. For instance, 4-HBA can be used to prepare liquid-crystalline polymer (e.g., US2012/190813). Furthermore, 4-HBA has been shown to possess anti-angiogenic, anti-inflammatory and anti-nociceptive activities and, for instance, it has been patented to treat ischemic brain disease (WO2005/030189).

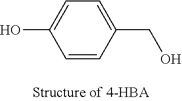

The global market for vanillin, the world's most popular flavor, for which 4-HBA is a precursor, is estimated to be between 15-16,000 tons per year.

The market for 4-HBA is thus extensive covering the production of food, polymers and pharmaceutical compounds. Different quality grades of 4-HBA are required, notably for medical applications. However, the production of 4-HBA remains difficult.

An important goal in synthetic chemistry is to develop environmentally friendly and increasingly safer processes. HBA's are generally synthesized by reduction of the corresponding aromatic aldehydes. 4-HBA is industrially produced by the reaction of phenol with formaldehyde in the presence of a basic catalyst. Through this process, a mixture of 4-HBA (p-hydroxybenzyl alcohol) and o-hydroxybenzyl alcohol is obtained and the two isomers have to be separated. The o-hydroxybenzyl alcohol is predominantly formed and the addition of various solvents is commonly used to increase the amount of 4-HBA produced. The isolation of the pure compounds from these reaction mixtures is further complicated by the formation of by-products. Indeed, not only these two isomers are produced but, as a result of their high reactivity, the hydroxybenzyl alcohols react with the formaldehyde present in the reaction mixture and self-condensation can also occur.

Many improvements have been introduced to this process (U.S. Pat. No. 4,205,188), notably the addition of catalyst (U.S. Pat. No. 5,019,656). However, it still requires the use of large amounts of organic compounds and solvent and involves several purification steps.

These problems notwithstanding, few biotechnological alternatives have been developed to safely produce renewable 4-HBA within the frame of a ‘green chemistry’ approach.

Bacterial production of 4-HBA has been described recently, involving either an increased production of aromatic amino acids or a reduction of their use by the host cell. A bacterial cell, which has an increased flux in the biosynthesis of one or more aromatic amino acids has been disclosed (EP1764415). Major disadvantages of this approach include the need to purify 4-HBA from the bacteria metabolites and its empirical nature, with no biosynthetic pathway clearly identified.

Alternatively, several biosynthetic pathways have been disclosed that, theoretically, could lead to the production of 4-HBA. However, they have not been disclosed as such and it is not clear whether 4-HBA could be really isolated therefrom.

In plants, the chain shortening of p-coumaric acid to p-hydroxybenzaldehyde has been disclosed in Vanilla planifolia (US2003/0070188).

In bacteria, a method for the production of p-hydroxybenzoate in species of Pseudomonas and Agrobacterium has been disclosed (EP1292682). The p-cresol methylhydroxylase (PCMH) converts p-cresol to 4-HBA and further oxidizes it to p-hydroxybenzoate.

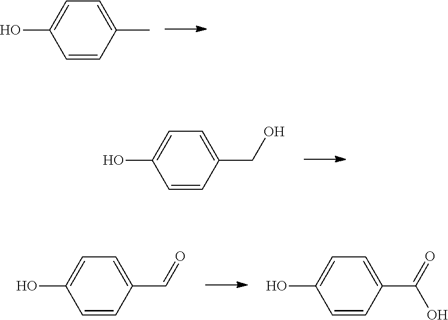

Reaction Catalyzed by PCMH

p-Cresol has problems in term of stability and toxicity. But most importantly, PCMH uses 4-HBA as a substrate and further oxidizes it making it unsuitable for 4-HBA production. Thus, the preparation of 4-HBA by this process would involve its costly regeneration from p-hydroxybenzoate.

In conclusion, there is an urgent need for a new method for producing efficiently 4-HBA, while both limiting the use of organic solvents and facilitating its purification.

### SUMMARY OF THE INVENTION

The present invention relates to the discovery that a ThiH (tyrosine lyase) enzyme from the thermophilic bacterium Moorella thermoacetica having the amino acid sequence of SEQ ID No 2 is capable of producing 4-HBA from tyrosine in an efficient manner. Its ability to catalyze this reaction is really surprising because the well-know homologous ThiH enzyme from E coli has been reported to be unable to produce 4-HBA (Kriek et al. 2007 Angew Chem Int Ed Engl. 2007; 46(48):9223-6). Thus, the use of this enzyme represents a novel way to produce 4-HBA through safer and sustainable production processes involving only one step from tyrosine.

It is thus provided an isolated or recombinant enzyme comprising an amino sequence having at least 80% identity with SEQ ID No 2 and being capable of producing 4-HBA and p-cresol from L-tyrosine. Preferably, the enzyme comprises or consists of an amino sequence having at least 90, 95, 97.5 or 99% identity with SEQ ID No 2. More particularly, the enzyme may comprise or consist of the amino sequence of SEQ ID No 2.

It is also provided a composition or a kit comprising the isolated or recombinant enzyme as defined above and a solid support on which is immobilized the enzyme as defined above. In particular, the composition may include iron and sulfur as enzyme additives and a reducing agent such as dithiothreitol or beta-mercaptoethanol. In addition the composition may include S-adenosyl L-methionine (SAM), the enzyme cofactor or methionine and ATP when in the presence of SAM synthase.

It is further provided a recombinant nucleic acid construct or vector comprising a nucleic acid sequence encoding the enzyme as defined above. More particularly, the nucleic acid construct or vector is suitable for expressing the said enzyme. In addition, it is provided a recombinant host cell comprising a nucleic acid, a recombinant nucleic acid construct or a recombinant vector comprising a nucleic acid sequence encoding the enzyme as defined above.

It is provided a method for producing an enzyme capable of making 4-HBA and p-cresol from L-tyrosine, comprising culturing the host cell as defined above, under conditions conducive to the production of the enzyme, and recovering and/or purifying the enzyme. Alternatively, it is also provided a method for producing an enzyme capable of making 4-HBA and p-cresol from L-tyrosine, comprising the in vitro expression of the enzyme with a nucleic acid encoding the enzyme as defined above. Optionally, the method further comprises a step of immobilizing the enzyme on a solid support.

The present invention also relates to the use of an enzyme as defined above, a composition, kit or solid support comprising the enzyme, or a recombinant host cell comprising a nucleic acid, a recombinant nucleic acid construct or a recombinant vector comprising a nucleic acid sequence encoding the enzyme as defined above, for producing 4-hydroxyl benzyl alcohol (4-HBA) or an analog thereof.

Accordingly, the present invention relates to a method for producing 4-hydroxyl benzyl alcohol (4-HBA) or an analog thereof comprising contacting tyrosine or an analog thereof with an enzyme comprising an amino sequence having at least 80% identity with SEQ ID No 2 and being capable of producing 4-HBA and p-cresol from L-tyrosine, and optionally recovering 4-HBA or the analog thereof.

The present invention also relates to a method for producing 4-hydroxyl benzyl alcohol (4-HBA) or an analog thereof comprising culturing a recombinant host cell comprising a nucleic acid, a recombinant nucleic acid construct or a recombinant vector comprising a nucleic acid sequence encoding the enzyme comprising an amino sequence having at least 80% identity with SEQ ID No 2 and being capable of producing 4-HBA and p-cresol from L-tyrosine in a medium comprising tyrosine or an analog thereof, and optionally recovering 4-HBA or the analog thereof.

Finally, the present invention relates to a method for producing a compound of interest, comprising producing 4-HBA or an analog thereof by the method according to the present disclosure and using the 4-HBA or the analog thereof for producing the compound of interest. Optionally, the compound of interest is selected from the group consisting of p-hydroxybenzaldehyde, p-hydroxybenzoic acid, bisoprolol, 4,4′-dihydroxydiphenylmethane, vanillin and polymers, especially liquid-crystalline polymer.

### BRIEF DESCRIPTION OF THE FIGURES

FIG. 1: Enzymes purification analyzed by SDS PAGE. ThiH from (a) Moorella thermoacetica (ThiH_{MO}), (b) Carboxydothermus hydrogenoformans (ThiHcH), Chlorobium tepidum (ThiHcT), Clostridum acetobutylicum (ThiHcA) and (c) ThiH from Escherichia coli (ThiHEC).

FIG. 2: ThiH activity with tyrosine as substrate under anaerobic and reducing conditions in the presence of S-adenosyl L-methionine (SAM) and dithionite from (a) Clostridum acetobutylicum, (b) Chlorobium tepidum, (c) Escherichia coli, (d) Carboxydothermus hydrogenoformans, (e) Moorella thermoacetica analyzed by HPLC compared to (f) reference compounds. Reactions were performed and analyzed (1) in the absence of tyrosine or with a full reaction medium at initial (2) and final (3) reaction times.

FIG. 3: NMR spectroscopy analysis of the enzymatic reaction with ThiH_{MO} (a) in the presence of ¹³C-labelled tyrosine showing the formation of 4-HBA. ¹³C-NMR analysis of the reaction with ThiH_{MO} (upper trace) and reference spectrum of ¹³C-tyrosine (lower trace). (b) Reference ¹³C-NMR spectrum of 4-hydroxy benzyl alcohol (4-HBA).

FIG. 4: C₁₈ HPLC analysis of the reaction of ThiH_{MO} under anaerobic conditions after 12 h of incubations at 25° C. (275 nm)—The reaction was performed with (1) ThiH_{MO} (40 μM), SAM (1 mM), tyrosine (1 mM) and sodium dithionite as one-electron donor (2 mM) in Tris buffer pH 7.5 or in the absence of (2) sodium dithionite, (3) SAM or (4) ThiH_{MO}. SAM degradation products such as adenine (Ad) or methylthioadenosine (MTA) are formed independently of the enzymatic reaction.

FIG. 5: pH-dependent activity of ThiH_{MO} analyzed by HPLC and fluorescence. ThiH (40 μM) was incubated under anaerobic and reducing conditions in the presence of S-adenosyl L-methionine (1 mM), dithionite (2 mM) and tyrosine (1 mM).

FIG. 6: Mass fragmentation of standard 4-HBA.

FIG. 7: LC-MS³ analysis of (FIG. 7A) standard 4-HBA and (FIG. 7B) Minimal medium after growth of E. coli expressing ThiH from Moorella Thermoacetica.

FIG. 8: HPLC analysis of minimal medium after growth of E. coli BL21 harboring (A) an empty plasmid or (B) ThiH from Moorella Thermoacetica.

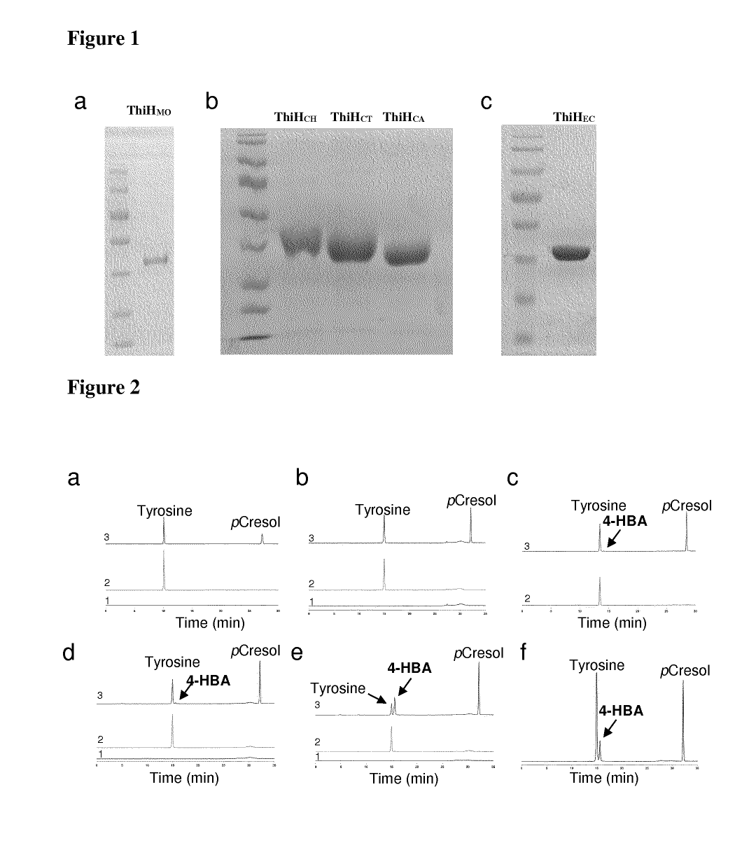

Table 1—¹³C-NMR chemical shifts of tyrosine and p-cresol and measured ¹³C-NMR chemical shifts of ¹³C-labelled tyrosine, glycine, glyoxylate hydrate and 4-HBA in the mixture. CH₂ of tyrosine was set at 57. ppm.

### DETAILED DESCRIPTION OF THE INVENTION

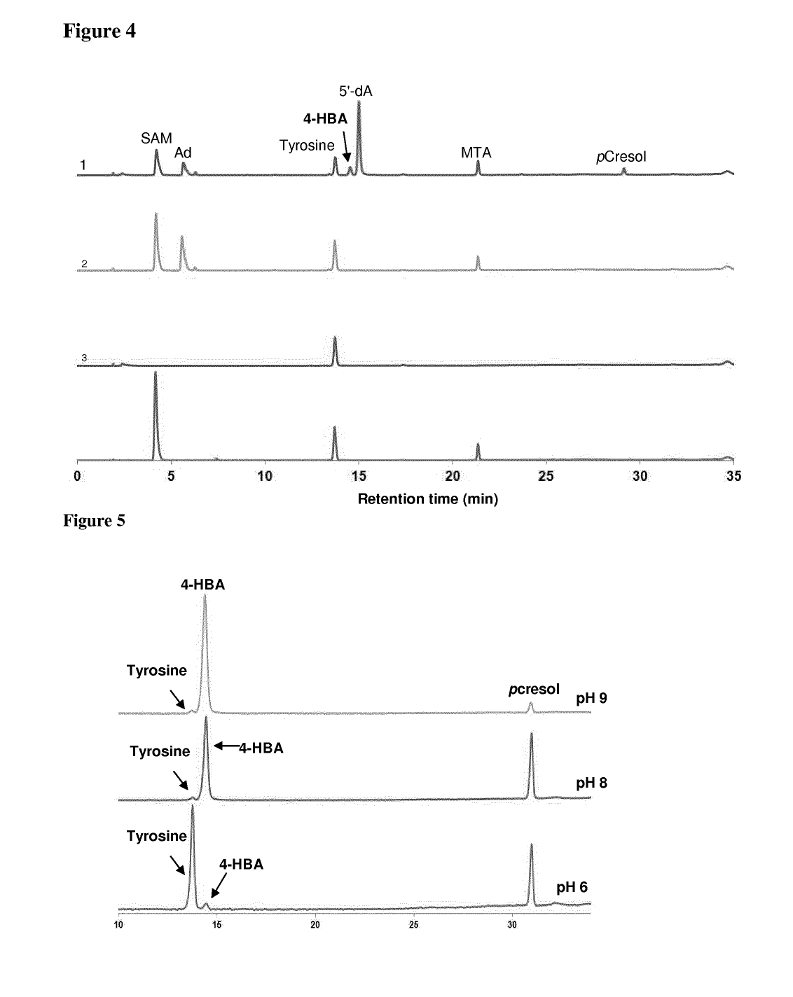

The inventors surprisingly identified an enzyme, which specifically converts the amino acid tyrosine into p-cresol and 4-HBA (FIGS. 2-4 and 6-8 and Table 1). Although p-cresol is not of particular interest, its properties make the purification process of 4-HBA very straightforward.

Furthermore, the p-cresol/4-HBA ratio can be modified by the reaction conditions. Notably, a basic pH, preferably in the range of pH 7-10, improves the yield of 4-HBA and should be preferentially chosen. The reaction can be performed with standard enzyme buffers including non-exclusively phosphate, Tris and Borax buffers (FIG. 5).

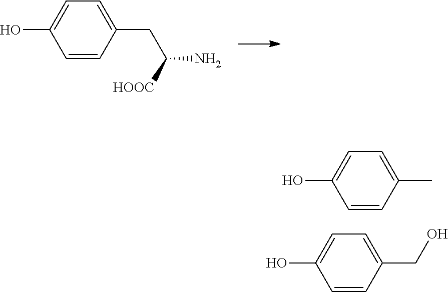

Reaction Catalyzed by the Enzyme According to the Present Invention.

Furthermore, contrary to the standard chemical processes, this enzymatic synthesis does not lead to different HBAs or side products.

It is thus possible to produce 4-HBA in one step, without any organic solvent or toxic chemicals contrary to the currently available industrial processes. In summary: (a) ThiH_{MO} allows for a totally sustainable production of 4-HBA and (b) this enzyme produces 4-HBA safely, notably for medical and food applications.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding an enzyme of the present invention. Control sequences may be native (i.e., from the same gene) or heterologous (i.e., from a different gene and/or a different species) to the polynucleotide encoding the enzyme. Preferably, control sequences are heterologous. Well-known control sequences and currently used by the person skilled in the art will be preferred. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding the enzyme. The functional combination of control sequences and coding sequences can be referred as expression cassette.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding the enzyme of the invention and is operably linked to control sequences that provide for its expression. Then the expression vector comprises an expression cassette suitable for expressing the enzyme of the invention.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance).

Recombinant: Recombinant refers to a nucleic acid construct, a vector and a protein produced by genetic engineering.

Heterologous: in the context of a host cell, a vector or a nucleic acid construct, it designates a coding sequence for the enzyme introduced into the host cell, the vector or the nucleic acid construct by genetic engineering. In the context of a host cell, it can mean that the coding sequence for the enzyme originates from a source different from the cell in which it is introduced. Alternatively, it can also mean that the coding sequence for the enzyme comes from the same species as the cell in which it is introduced but it is considered heterologous due to its environment which is not natural, for example because it is under the control of a promoter which is not its natural promoter, or is introduced at a location which differs from its natural location.

Nucleic acid construct: The term “nucleic acid construct” means a nucleic acid molecule, either single- or double-stranded, which is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to a coding sequence, in such a way that the control sequence directs expression of the coding sequence.

Sequence identity: The sequence identity between two amino acid sequences is described by the parameter “sequence identity”. For purposes of the present invention, the “percentage identity” between two amino acid sequences (A) and (B) is determined by comparing the two sequences aligned in an optimal manner, through a window of comparison. Said alignment of sequences can be carried out by well-known methods, for example, using the algorithm for global alignment of Needleman-Wunsch. Protein analysis software matches similar sequences using measures of similarity assigned to various substitutions, deletions and other modifications, including conservative amino acid substitutions. Once the total alignment is obtained, the percentage of identity can be obtained by dividing the full number of identical amino acid residues aligned by the full number of residues contained in the longest sequence between the sequence (A) and (B).

Sequence identity is typically determined using sequence analysis software. For comparing two amino acid sequences, one can use, for example, the tool “Emboss needle” for pairwise sequence alignment of proteins providing by EMBL-EBI and available on: www.ebi.ac.uk/Tools/services/web/toolform.ebi?tool=emboss_needle&context=protein, using default settings: (I) Matrix: BLOSUM62, (ii) Gap open: 10, (iii) gap extend: 0.5, (iv) output format: pair, (v) end gap penalty: false, (vi) end gap open: 10, (vii) end gap extend: 0.5.

Variant: The term “variant” means an enzyme capable of producing 4-HBA and p-cresol from L-tyrosine and comprising an alteration, i.e., a substitution, insertion, and/or deletion, at one or more (e.g., several) positions. In particular, the variant may have alterations at not more than 11, 10, 9, 8, 7, 6, 5, 4, 3, 2 or 1 amino acids, e.g., may have substitution, insertion, and/or deletion of 1, 2, 3, 4, 5, 6, 7, 8, 9 or 10 amino acids. A substitution means replacement of the amino acid occupying a position with a different amino acid; a deletion means removal of the amino acid occupying a position; and an insertion means adding an amino acid adjacent to and immediately following the amino acid occupying a position. The substitution can be a conservative substitution. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill (1979, In, The Proteins, Academic Press, New York). Common substitutions are the followings Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, LeuA al, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like. Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for the capacity to produce 4-HBA from L-tyrosine to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for instance, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner ei a/., 1988, DNA 7: 127). Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Tyrosine analogs: The tyrosine analogs refer to any analog of tyrosine capable of being converted by the enzyme of the invention. In particular, a tyrosine analog can be an analog of D-tyrosine, L-tyrosine or DL-tyrosine, preferably L-tyrosine. Preferably, the tyrosine analog has one or two substituents on the hydrobenzyl moiety or is an isomer of tyrosine. For instance, the tyrosine analog can be selected in the group consisting of the compounds O-Methyl-D-tyrosine (CAS No 39878-65-4), O-Methyl-L-tyrosine (CAS No 6230-11-1), O-Methyl-DL-tyrosine, O-Benzyl-D-tyrosine (CAS No 65733-15-5), O-Benzyl-L-tyrosine (CAS No 16652-64-5), O-Acetyl-L-tyrosine (CAS No 6636-22-2), O-2,6-Dichlorobenzyl-D-tyrosine, O-2,6-Dichlorobenzyl-L-tyrosine (CAS No 40298-69-9), O-tert-Butyl-D-tyrosine (CAS No 186698-58-8), O-tert-Butyl-L-tyrosine (CAS No 18822-59-8), L-meta-Tyrosine (CAS No 587-33-7), D-meta-Tyrosine (CAS No 32140-49-1), DL-meta-Tyrosine, DL-o-Tyrosine (CAS No 2370-61-8), L-2-Hydroxyphenylalanine (CAS No 7423-92-9), m-Iodo-L-tyrosine (CAS No 70-78-0), O-Phospho-L-tyrosine (CAS No 21820-51-9), 3,5-Diiodo-D-tyrosine (CAS No 16711-71-0), 3,5-Diiodo-L-tyrosine (CAS No 300-39-0), 3,5-Dinitro-D-tyrosine (CAS No 779321-23-2), 3,5-Dinitro-L-tyrosine (CAS No 17360-11-1), 3-Amino-L-tyrosine (CAS No 23279-22-3), 3-Chloro-D-tyrosine (CAS No 162599-96-4), 3-Chloro-L-tyrosine (CAS No 7423-93-0), 3-Fluoro-DL-tyrosine (CAS No 139-26-4), 3-Iodo-D-tyrosine (CAS No 25799-58-0), 3-Nitro-D-tyrosine (CAS No 32988-39-9), 3-Nitro-L-tyrosine (CAS No 621-44-3), D-3,5-Dibromotyrosine (CAS No 50299-42-8), L-3,5-Dibromotyrosine (CAS No 300-38-9), L-Homotyrosine (CAS No 141899-12-9), and D-Homotyrosine (CAS No 185617-14-5), preferably in the group consisting of the compounds O-Methyl-L-tyrosine (CAS No 6230-11-1), O-Benzyl-L-tyrosine (CAS No 16652-64-5), O-Acetyl-L-tyrosine (CAS No 6636-22-2), OO-2,6-Dichlorobenzyl-L-tyrosine (CAS No 40298-69-9), O-tert-Butyl-L-tyrosine (CAS No 18822-59-8), L-meta-Tyrosine (CAS No 587-33-7), L-2-Hydroxyphenylalanine (CAS No 7423-92-9), m-Iodo-L-tyrosine (CAS No 70-78-0), O-Phospho-L-tyrosine (CAS No 21820-51-9), 3,5-Diiodo-L-tyrosine (CAS No 300-39-0), 3,5-Dinitro-L-tyrosine (CAS No 17360-11-1), 3-Amino-L-tyrosine (CAS No 23279-22-3), 3-Chloro-L-tyrosine (CAS No 7423-93-0), 3-Nitro-L-tyrosine (CAS No 621-44-3), L-3,5-Dibromotyrosine (CAS No 300-38-9), and L-Homotyrosine (CAS No 141899-12-9. These analogs are commercially available, for instance at Chem-Impex International Inc.

Enzyme

It is provided an enzyme capable of producing 4-HBA and p-cresol in presence of L-tyrosine. Indeed, the inventors identified a tyrosine lyase ThiH from Moorella thermoacetica having the amino acid sequence of SEQ ID No 2. The enzyme is surprisingly capable of producing 4-HBA and p-cresol in presence of L-tyrosine and the co-factor S-adenosyl-L-methionine (SAM) following the reaction:

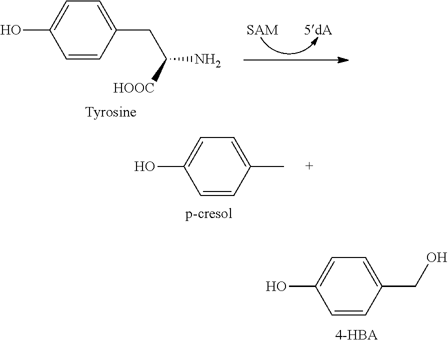

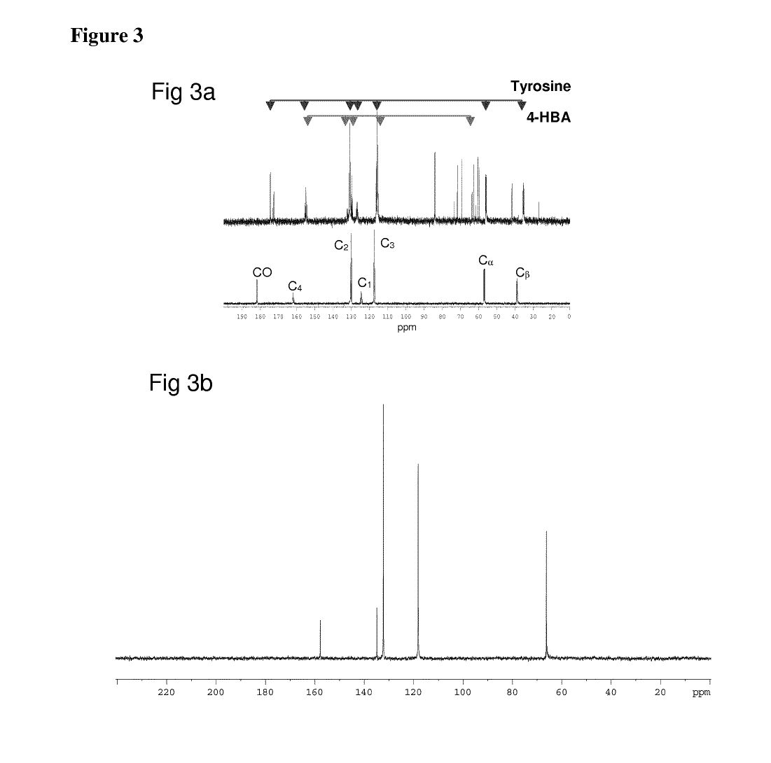

NMR analysis of the reaction demonstrated, starting from tyrosine, that the enzyme produces besides the expected molecules i.e. glyoxylate, p-cresol and glycine and a novel compound: 4-HBA, which has never been reported for such type of enzymes (FIG. 3).

The p-cresol/4-HBA ratio is influenced by the reaction conditions; notably the pH affects strongly this ratio. To favor 4-HBA production, the pH should be between pH 7 and 10, as illustrated in FIG. 5.

Therefore, it is provided an isolated or recombinant enzyme capable of producing 4-HBA and p-cresol in presence of L-tyrosine and comprising an amino acid sequence having at least 60% identity with SEQ ID No 2. Preferably, the isolated or recombinant enzyme comprises or consists of an amino acid sequence having at least 80, 85, 90, 95, 97, 98, 99% identity with SEQ ID No 2. In a very particular aspect, the isolated or recombinant enzyme comprises or consists of the amino acid sequence of SEQ ID No 2.

Because of their homologies with ThiH, the other radical SAM tyrosine lyases, CofH (26.9% similarity) involved in the biosynthesis of F420 cofactor (Decamps et al. (2012) J Am Chem Soc 134, 18173-18176.) and HydG (44.2% similarity) involved in the H-cluster biosynthesis (Nicolet et al. (2009) FEBS Lett.; 584(19):4197-202.), are also likely to be able to produce 4-HBA, either naturally or through enzyme engineering.

A method for testing the capacity of an enzyme to produce 4-HBA from L-tyrosine is for instance disclosed in details in the example section. More specifically, the enzyme is contacted with L-tyrosine in presence of the co-factor S-adenosyl-L-methionine (SAM) and the production of 4-HBA is detected. More particularly, the enzyme is capable of producing 4-HBA and p-cresol with a ratio ranging from between 1:30 to 30:1, preferably between 1:10 to 10:1, still more preferably between 2:3 and 3:2.

Based on the teaching of the present disclosure, the one skilled in the art can identify other enzymes from microorganisms having the 4-HBA producing activity from L-tyrosine. The polypeptide may be identified and obtained from other sources including microorganisms isolated from nature (e.g., soil, composts, water, etc.) or DNA samples obtained directly from natural materials (e.g., soil, composts, water, etc.) using the probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected, the polynucleotide can be isolated or cloned by utilizing techniques that are known to those of ordinary skill in the art (see, e.g., Sambrook et al., 1989). In addition, the person skilled in the art can prepare variants of the ThiH from Moorella thermoacetica having the amino acid sequence of SEQ ID No 2 by currently used methods. In particular, variants with advantageous properties such as an increased stability (e.g., thermostability), increased production of 4-HBA relative to p-cresol (e.g., improved ratio of 4-HBA/p-cresol).

It is also provided a hybrid polypeptide or fusion polypeptide in which the amino acid sequence of the enzyme as defined above is fused at the N-terminus or the C-terminus of a region of another polypeptide. Techniques for producing fusion polypeptides are known in the art, and include ligating the coding sequences encoding the enzyme and the addition region of another polypeptide so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

The addition region of the fusion polypeptide can be selected in order to enhance the stability of the enzyme according to the present disclosure, to promote the secretion (such as a N-terminal hydrophobic signal peptide) of the fusion protein from a cell (such as a bacterial cell or a yeast cell), or to assist in the purification of the fusion protein. More particularly, the additional region can be a tag useful for purification or immobilization of the enzyme. Such a tag is well-known by the person skilled in the art, for instance a His tag (His6), a FLAG tag, a HA tag (epitope derived from the Influenza protein haemagglutinin), a maltose-binding protein (MPB), a MYC tag (epitope derived from the human proto-oncoprotein MYC), a STREP tag or a GST tag (small glutathione-S-transferase).

A fusion polypeptide can further comprise a cleavage site between the enzyme and the addition region. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Nucleic Acid Constructs

The present invention relates to a polynucleotide encoding an enzyme of the present invention. The nucleic acid can be DNA (cDNA or gDNA), RNA, or a mixture of the two. It can be in single stranded form or in duplex form or a mixture of the two. It can comprise modified nucleotides, comprising for example a modified bond, a modified purine or pyrimidine base, or a modified sugar. It can be prepared by any method known to one skilled in the art, including chemical synthesis, recombination, and mutagenesis. In particular, such a polynucleotide is disclosed in SEQ ID No 1.

The present invention also relates to nucleic acid constructs comprising a polynucleotide encoding an enzyme according to the present disclosure operably linked to one or more control sequences that direct the expression of the coding sequence in a suitable host cell under conditions compatible with the control sequences. A polynucleotide may be manipulated in a variety of ways to provide for expression of the enzyme. Manipulation of the polynucleotide prior to its insertion into a vector may be desirable or necessary depending on the expression vector. The techniques for modifying polynucleotides utilizing recombinant DNA methods are well known in the art.

The control sequence may include a promoter that is recognized by a host cell or an in vitro expression system for expression of a polynucleotide encoding an enzyme of the present invention. The promoter contains transcriptional control sequences that mediate the expression of the enzyme. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylllA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989. Examples of tandem promoters are disclosed in WO 99/43835.

Examples of suitable promoters in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene; and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et ai, 1995, Journal of Bacteriology 177: 3465-3471).

The control sequence may also be a leader, a non-translated region of an mRNA that is important for translation by the host cell. The leader is operably linked to the 5′-terminus of the polynucleotide encoding the enzyme. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide encoding the enzyme and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of the enzyme and directs the enzyme into the cell's secretory pathway. The 5′-end of the coding sequence of the polynucleotide may inherently contain a signal peptide coding sequence naturally linked in translation reading frame with the segment of the coding sequence that encodes the enzyme. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 1 1837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

Expression Vectors

The present invention also relates to recombinant expression vectors comprising a nucleic acid construct as disclosed above, or a polynucleotide encoding an enzyme of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the enzyme at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophy, and the like.

Examples of bacterial selectable markers are Bacillus licheniformis or Bacillus subtilis genes or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus gene.

The vector preferably contains an element(s) that permits integration of the vector into the host cell's genome or autonomous replication of the vector in the cell independent of the genome.

When integration into the host cell genome occurs, integration of the sequences into the genome may rely on homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo. Examples of bacterial origins of replication are the origins of replication of plasmids pBR322, pUC19, pACYC177, and pACYC184 permitting replication in E. coli, and pUB1 10, pE194, pTA1060, and pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a polynucleotide encoding the enzyme according to the present disclosure operably linked to one or more control sequences that direct the production of the enzyme of the present invention. A construct or vector comprising a polynucleotide encoding the enzyme of according to the present disclosure is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma. The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. The bacterial host cell may also be any Streptococcus cell including, but not limited to, Streptococcus equisimilis, Streptococcus pyogenes, Streptococcus uberis, Streptococcus equi and Streptococcus zooepidemicus cells. The bacterial host cell may further be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al, 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier ei a/., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell. The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK). The fungal host cell may be a yeast cell. “Yeast” as used herein includes ascosporogenous yeast (Endomycetales), basidiosporogenous yeast, and yeast belonging to the Fungi imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980). The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

The cell can also be a mammalian cell, for example COS, CHO (U.S. Pat. No. 4,889,803; U.S. Pat. No. 5,047,335). In a particular embodiment, the cell is non-human and non-embryonic. In addition, the enzyme of the invention could be produce by a non-human transgenic animal, for instance in the milk produces by the animal.

The cell can be a plant cell. Then, the enzyme of the invention could be produce by a transgenic plant.

A particular host cell of interest in the present disclosure is a host cell overproducing tyrosine or analog thereof, in particular L-tyrosine. In particular, the host cell can be a cell overproducing tyrosine, more preferably a genetically engineered host cell. Cells overproducing tyrosine are known. Several different microorganisms have been modified for L-Tyr production. Corynebacterium glutamicum, Arthrobacter globiformis, and Brevibacterium lactofermentum L-Tyr-overproducing strains were developed by classical mutagenesis methods (Ito et al., Agric Biol Chem. 1990 March; 54(3):699-705; Hagino, H., and K. Nakayama. 1973. Agric. Biol. Chem. 39:2013-2023; Roy, et al. 1997. J. Sci. Ind. Res. 56:727-733). Metabolic engineering and protein-directed evolution strategies have been used to construct E. coli L-Tyr-producing strains (US 2005/0277179; Liitke-Eversloh T, Stephanopoulos G. Appl Environ Microbiol. 2005 November; 71(11):7224-8; Liitke-Eversloh T, Stephanopoulos G. Appl Microbiol Biotechnol. 2007 May; 75(1):103-10; Patnaik Ret al. Biotechnol Bioeng. 2008 Mar. 1; 99(4):741-52; Chavez-Bejar et al, Appl Environ Microbiol. 2008 May; 74(10): 3284-3290). Therefore, a host cell overproducing tyrosine and expressing (or being able to express under suitable conditions) the enzyme according to the present disclosure is of particular interest.

Method of Enzyme Production

The present invention also relates to (a) methods of producing the enzyme of the present invention wherein a nucleic acid construct encoding the enzyme according to the present disclosure is expressed; and (b) recovering the enzyme.

In a first aspect, the present invention also relates to in vitro methods of producing the enzyme of the present invention wherein a nucleic acid construct as disclosed above is contacted with an in vitro expression system; and recovering the enzyme. The in vitro expression systems are well known to the person skilled in the art and are commercially available.

In a second aspect, the present invention also relates to methods of producing the enzyme of the present invention, comprising (a) culturing a cell, which in its wild-type form produces the enzyme according to the present disclosure, under conditions conducive for production of the enzyme; and (b) recovering the enzyme. In a preferred aspect, the cell is a Moorella thermoacetica cell. Moorella thermoacetica was previously known as Clostridium thermoaceticum.

In a third aspect, the present invention also relates to methods of producing the enzyme according to the present disclosure, comprising (a) cultivating a recombinant host cell as described above under conditions conducive for production of the enzyme; and (b) recovering the enzyme.

The host cells are cultivated in a nutrient medium suitable for production of polypeptides using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the enzyme to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the enzyme is secreted into the nutrient medium, the enzyme can be recovered directly from the medium. If the enzyme is not secreted, it can be recovered from cell lysates. The enzyme may be detected using methods known in the art that are specific for the enzyme. These detection methods include, but are not limited to, use of specific antibodies, detection of tag, formation of an enzyme product, or disappearance of an enzyme substrate. For example, an enzyme assay may be used to determine the activity of the enzyme.

The enzyme may be recovered using methods known in the art. For example, the enzyme may be recovered from the nutrient medium by conventional procedures including, but not limited to, collection, centrifugation, filtration, extraction, spray-drying, evaporation, or precipitation.

The enzyme may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides. In an alternative aspect, the enzyme is not recovered, but rather a host cell of the present invention expressing the enzyme is used as a source of the enzyme.

In addition, it is also provided the use of the enzyme according to the present disclosure for preparing the enzyme immobilized on a solid support; and a method for preparing an enzyme as disclosed above immobilized on a solid support comprising producing the enzyme as detailed above and immobilizing the enzyme on a solid support.

The present invention also relates to a solid support, the enzyme according to the present invention being immobilized on the solid support. Immobilization means are well-know to the person skilled in the art (‘Enzyme Technology’ by Martin Chaplin and Christopher Bucke (Cambridge University Press, 1990); Lim et al. 2009, Process Biochemistry 44, 822-828; WO2011/040708; Alloue et al, Biotechnol Agron Soc Environ 2008, 12, 57-68; the disclosure thereof being incorporated herein by reference. The enzyme according to the present disclosure can be immobilized on the solid support by any convenient mean, in particular adsorption adsorption, covalent binding, entrapment or membrane confinement. A wide variety of insoluble materials may be used to immobilize the enzyme. These are usually inert polymeric or inorganic matrices. For example, the enzyme can be immobilized on a polyurethane matrix (Gordon et al., 1999, Chemical-Biological Interactions 14:463-470) on activated sepharose, alginate, amberlite resin, Sephadex resin or Duolite resin. Other solid supports useful for the invention include resins with an acrylic type structure, polystyrene resins, macroreticular resins and resins with basic functional groups, such as Sepabeads EC-EP and Relizime (Resindion Srl, Mitsubishi Chemical Corporation) and Eupergit C (Röhm GmbH & Co. KG). In any case, the enzyme is brought in contact with the resin and is either immobilized through the high reactivity of the functional groups or activation of the resin with a bifunctional agent, such as glutaraldehyde, so as to bind the enzyme to the matrix, or is absorbed on the resin and then stabilized by cross-linking with a bifunctional agent (glutaraldehyde). The solid support can be for instance membranous, particulate or fibrous. More particularly, the solid support is preferably a bead, e.g., micro- or nanobeads. Then, the enzyme is immobilized on a solid support in order to prepare a reactor, which can be for instance an enzyme reactor, a membrane reactor, a continuous flow reactor such as a stirred tank reactor, a continuously operated packed bed reactor, or a continuously operated fluidized bed reactor, or a packed bed reactor.

Compositions and Kits

The produced enzyme can be formulated in a composition. The composition comprises components suitable for enzyme preservation. The enzyme can be free or immobilized on a solid support, preferably beads. The composition can be liquid or dry. It comprises the enzyme according to the disclosure in a purified or enriched form. Liquid compositions preferably contain the enzyme in a purified or enriched form. However, auxiliaries such as a stabilizer like glycerol (also called glycerine), sorbitol or monopropylene glycol, additives like salts, sugar, preservatives, agents for to adjust the pH value (buffer), a redox agent such as DTT (dithiothreitol), or a sequester such as EDTA (ethylenediaminetetraacetic acid) can be added. In particular, the liquid composition can comprise at least 10, 20, 30, 40 or 50% (w/v) of glycerol sorbitol or monopropylene glycol, preferably between 20 and 50% (w/v). Preferably, the composition comprises glycerol. Optionally, the composition may further include the co-factor SAM. Typical liquid compositions are aqueous or oleaginous suspensions.

Therefore the present invention relates to a composition, especially an enzymatic composition, comprising the enzyme according to the present disclosure and appropriate auxiliaries, in particular those disclosed above. Preferably, the composition comprises, as enzymes or proteins component, at least 75, 80, 85, 90, 95% enzyme.

It is also provided a kit for producing 4-HBA comprising an enzyme, a composition, a support solid with the immobilized enzyme or a host cell capable of expressing the enzyme as described above. The kit may further comprise other reagents such as SAM, buffer and a reducing agent: a source of one-electron donor such as sodium dithionite, methyl viologen or an enzymatic systems such as flavodoxin/flavodoxin reductase/NADPH, and addition of iron and sulfur if necessary.

Methods and Uses

The present invention relates to the use of
the enzyme according to the present disclosure; or
the solid support with the immobilized enzyme; or
the host cell capable of expressing the enzyme; or
a kit as disclosed above;

for producing 4-HBA or an analog thereof, or a compound of interest prepared from 4-HBA or the analog thereof, preferably for producing 4-HBA or a compound of interest prepared from 4-HBA.

It also relates to a method for producing 4-HBA or an analog thereof comprising contacting tyrosine or an analog thereof with an enzyme comprising an amino sequence having at least 80% identity with SEQ ID No 2 and being capable of producing 4-HBA and p-cresol from L-tyrosine, and optionally recovering 4-HBA or the analog thereof.

It further relates to a method for producing 4-hydroxyl benzyl alcohol (4-HBA) or an analog thereof comprising culturing a recombinant host cell expressing an enzyme comprising an amino sequence having at least 80% identity with SEQ ID No 2 and being capable of producing 4-HBA and p-cresol from L-tyrosine in a medium comprising tyrosine or an analog thereof, and optionally recovering 4-HBA or the analog thereof. Optionally, 4-HBA or the analog thereof can be recovered from the culture medium.

Preferably, the tyrosine or an analog thereof has the following formula:

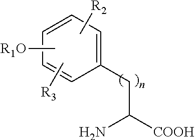

wherein n is 0, 1 or 2, preferably 1

R1 is selected from the group consisting of a hydrogen, a C1-C4 alkyl, an aryl, an C1-C3alkylaryl, a C1-C4 acyl, and a phosphate, preferably from the group consisting of methyl, ethyl, t-butyl, phenyl, benzyl and acetyl;

and R2 and R3, independently from each other, can be selected from the group consisting of a hydrogen, a halogen (preferably chloro, iodo, bromo or fluroro), a C1-C4 alkyloxy (preferably methoxy or ethoxy), nitro, cyano, amino, amide, and trifluoromethyl.

The tyrosine or the analog can be L or D, preferably L.

Preferably, the 4-HBA and an analog thereof has the following formula

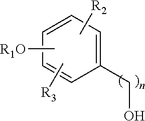

wherein n, R1, R2 and R3 have the same definition as above.

OR1 can be in position ortho, meta or para. Preferably, OR1 is in para.

Preferably, R2 and/or R3 are in position meta.

More particularly, the tyrosine or an analog thereof has the following formula:

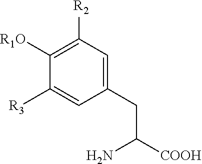

and the 4-HBA or an analog thereof has the following formula

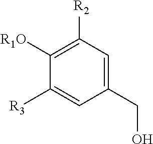

wherein R1, R2 and R3 have the same definition than above.

In a preferred and particular embodiment, R1, R2 and R3 are hydrogen atoms.

Preferably, the tyrosine or an analog thereof is contacted with the enzyme in the presence of the SAM cofactor. The reaction, for in vitro production, is preferably performed under anaerobic and reducing conditions between pH 6 and 10. A source of one-electron donor will be preferably present, for instance, but not limited to, chemical agents such as dithionite, methyl viologen or enzymatic systems such as flavodoxin/flavodoxin reductase/NADPH. The reaction is preferentially performed between 20° C. and 40° C. but higher or lower temperatures might be used. The standard reaction is performed with ThiH_{MO} (40 μM), SAM (1 mM), tyrosine (1 mM), dithiothreitol (6 mM) and sodium dithionite (2 mM) in Tris buffer pH 8 under anaerobic conditions.

The method may comprise a further step of purification of the 4-HBA or the analog thereof. More specifically, 4-HBA and the by-product p-cresol can be easily separated in order to recover/purify 4-HBa. Indeed, the two compounds have very different hydrophobicity. They can be separated by any convenient method well known to the skilled person, for instance hydrophobic interaction chromatography (HIC), solid phase extraction (SPE), or distillation.

It is also provided an alternative method for producing 4-HBA comprising culturing a host cell as defined above, preferably a host cell overproducing tyrosine, and optionally recovering 4-HBA.

The present invention further relates to a method for preparing a compound of interest that comprises the production of 4-HBA, or an analog thereof, by a method according to the present invention and using the 4-HBA or the analog thereof for preparing the compound of interest. Such compound of interest is any compound that can be prepared from 4-HBA or an analog thereof, but preferably from 4-HBA. For instance, the compound of interest could be p-hydroxybenzaldehyde and p-hydroxybenzoic acid by 4-HBA oxidation (Garade et al. (2001) Catalysis Communications 10 (2009) 485-489), bisoprolol (WO2007/069266), 4,4′-dihydroxydiphenylmethane or polymers, especially liquid-crystalline polymer (e.g., US2012/190813) by condensation, or vanillin. In addition, as 4-HBA is of therapeutically interest, a formulation of 4-HBA can be also prepared such as a p-hydroxybenzyl alcohol-containing biodegradable polyoxalate nanoparticulate antioxidant (Kim et al, Biomaterials, 2011, 32(11):3021-9).

### EXAMPLES

### ThiH Cloning

Genes coding for tyrosine lyases (ThiH) variants from different organisms i.e. Moorella thermoacetica (MO), Carboxythermus hydrogenoformans (CH), Escherichia coli, Clostridium acetobutylicum (CA) and Chlorobium tepidum, were either cloned or synthesized and inserted into a suitable expression vector. Sequence-optimized synthetic genes of ThiH_{MO}, ThiHcH and ThiHcA were obtained from GenScript™ and were inserted into a pET-15b (Novagen®) vector between NdeI and BamHI restriction sites. The Thih_{CT} gene was amplified by a standard PCR protocol using 5′-GGTAATCCATATGATTGCGCTGCCCGCATGGCTGACC-3′ (SEQ ID No 11) and 5′-GGGAATTCTTATCACGTGCACTCCTCTGCGGGCAGG-3′ (SEQ ID No 12) oligonucleotides as primers and Phusion™ as polymerase. The amplified fragment was subsequently inserted into a pET-28a vector (Novagen®) between NdeI and EcoRI restriction sites. Thih_{EC}, was cloned using standard PCR protocols and inserted into a pASK-=17plus vector. The integrity of the cloned sequences was determined by sequencing the entire genes.

ThiH Expression and Purification.

E. coli BL21(DE3) cells were transformed with pET15b-ThiH (or pET28a-TiH or pASK17plus-ThiH) and grown aerobically overnight at 37° C. in LB medium supplemented with ampicillin (100 μg·mL-1). An overnight culture was then used to inoculate fresh LB medium supplemented with the same antibiotic and bacterial growth proceeded at 37° C. until the OD₆₀₀ reached 0.6. The cells were induced by adding 200 μM IPTG and collected after overnight growth at 20° C. After re-suspension in Tris-buffer (50 mM Tris, 300 mM KCl, 10 mM MgCl₂, 500 mM NaCl, pH 7.5), the cells were disrupted by sonication and centrifuged at 220,000×g at 4° C. for 90 minutes. The solution was then loaded onto a Ni-NTA Sepharose column previously equilibrated with Tris-buffer. The column was washed extensively with the same buffer. Three elution steps were performed at 25 mM, 75 mM and 500 mM imidazole in Tris-buffer. The over-expressed protein was eluted in the 500 mM imidazole fraction. Fractions containing ThiH were immediately desalted on a PD10 column (GE Healthcare) with Tris-buffer as eluent, concentrated in Amicon Ultra-4 (Millipore) with a molecular cut-off of 10 kDa and frozen in liquid nitrogen.

For ThiH expressed with the pASK17plus plasmid, a similar protocol was used but cells were induced by 200 μg·L⁻¹ anhydrotetracycline and the enzyme was purified using a Strep-Tactin resin equilibrated with Tris-buffer (50 mM Tris, 300 mM KCl, 10 mM MgCl₂, 500 mM NaCl, pH 7.5). Elution was performed using the same buffer containing 3 mM dethiobiotin.

Protein concentrations were determined by the Bradford protein assay, using BSA as a standard. The collected fractions were analyzed by 12% polyacrylamide gel electrophoresis under denaturing conditions (SDS-PAGE).

Reconstitution of Fe—S Clusters.

Reconstitution of Fe—S clusters was carried out anaerobically in a glove box (Bactron IV). Purified ThiH (170 μM monomer) was treated with 6 mM DTT and then incubated at 12° C. overnight with a 5-fold molar excess of both Na₂S (Fluka) and (NH₄)₂Fe(SO₄)₂ (Aldrich). The protein was desalted using a Sephadex G25 column (Amersham) and the colored fractions were concentrated with an Amicon Ultra-4 (Millipore). Protein concentrations were determined by the Bradford protein assay, using BSA as a standard. Iron concentrations were determined colorimetrically using bathophenanthroline under reducing conditions (Fish, W. W. (1988) Methods Enzymol 158, 357-64).

ThiH Enzymatic Assay.

The enzymatic assay was performed in an anaerobic glove box (Bactron IV) at 25° C. Samples contained 6 mM dithiothreitol, 3 mM sodium dithionite, 20 μM of reconstituted ThiH, along with 1 mM tyrosine and 1 mM SAM in Tris-buffer, pH 7.5. Control samples were prepared without enzyme to check tyrosine and SAM stability over time. Enzymatic assays were also performed using uniformly ¹³C-labeled tyrosine as substrate in the same conditions.

Reaction products were analyzed by HPLC using a C₁₈ column (LicroSphere, 5-μm, 4.6×150-mm) eluted at 1 mL/min with the following gradient: after a 1 ml step of Milli-Q H2O/0.1% trifluoroacetic acid, a three-step gradient from 0 to 9.6% in 17 min, from 9.6% to 35.2% in 7 min and finally from 35.2 to 42.4% acetonitrile with 0.1% TFA in 10 min was used to elute the samples. Detection was carried out at 257 nm and 275 nm with a photodiode array detector. SAM, 5′-deoxyadenosine, tyrosine, p-cresol and dihydroxybenzyl alcohol were injected as standards.

NMR Analysis.

¹³C-NMR chemical shifts of ¹³C-labelled tyrosine and its derivatives, p-cresol, glycine, glyoxylate hydrate and 4-HBA was determined using a Bruker AVANCE III 600 MHz spectrometer equipped with a 5 mm 1H/13C/15N/31P QCI Z-Gradient Cryoprobe. The 13C NMR spectra with proton decoupling were recorded with 64K data points using a spectral width of 36 000 Hz in the mixture. An exponential weighting function was applied prior to Fourier transformation. No internal reference was added. The CH₂ of tyrosine was set at 57 ppm in the reaction medium as in the pure sample.

Detection of 4-HBA as a Novel by-Product in ThiH.

UV-visible spectra of the five purified and in vitro Fe—S cluster reconstituted proteins exhibited typical Fe-to-S charge transfer bands at ˜320 and ˜420 nm consistent with the presence of one Fe₄S₄ center per polypeptide, as expected. These variants could be expressed and purified without ThiG in good yields contrary to the case of the E. coli enzyme. These five enzymes were assayed under identical conditions for tyrosine lyase activity. They were shown to catalyze efficient tyrosine cleavage and production of p-cresol in agreement with the current knowledge on ThiH enzymes. Unexpectedly, reacted mixtures from ThiH_{CH} and ThiH_{MO} exhibited an additional compound (compound 1) in the HPLC elution profile (rt ˜15.5 min), whose UV-visible and fluorescence spectra are consistent with a novel tyrosine derivative (FIG. 2). Using ThiH_{MO}, which produces the highest amount of compound 1, the inventors observed that its formation strictly depends on the presence of SAM, tyrosine and a reducing agent. Compounds 1 is thus produced by the radical-based activity of ThiH and not by an hypothetic secondary activity of the enzyme. Using ¹³C-labeled tyrosine as substrate and ThiH_{MO}, the inventors were able to perform ¹³C-NMR experiments on the whole reaction mixture. The ¹³C-NMR spectrum analysis indicates the presence of four major compounds: tyrosine, glyoxylate, and, unexpectedly, glycine and 4-HBA (see FIG. 3). The latter exhibits modified chemical shifts compared to tyrosine, notably at C4 (157 vs. 155 pm), C1 (133 vs 129 ppm) and a major shift on the Cβ (64 vs. 37 ppm) in full agreement with experimentally measured values for 4-hydroxy benzyl alcohol, used as a standard. In addition, commercially available 4-HBA displays a retention time on HPLC, as well as UV-visible and fluorescence properties identical to compound 1. This univocally demonstrates that compound 1 corresponds to 4-HBA produced by ThiH. Furthermore, 4-HBA synthesis proved to be independent of the reducing system used since the E. coli physiological reduction system, flavodoxin/flavodoxin reducatase/NADPH also allowed for efficient production of 4-HBA. In full agreement with previous reports on ThiH from E. coli (ThiHEC) the observed glyoxylate, results from the spontaneous hydrolysis of dehydroglycine, the precursor of the thiamine thiazole moiety. Its measured chemical shifts exactly matched those found for glyoxylate in the case of ThiHEC.

In the case of ThiH_{MO}, addition of 5′-dA, p-cresol or 4-HBA leads to a significant inhibition of the reaction. Also, addition of 4-HBA or p-cresol did not lead to any changes in their relative concentrations, excluding the inter-conversion of these molecules by the enzyme. On the other hand, assaying the enzyme under different pH conditions changes the 4-HBA/p-cresol ratio from 0.3 at pH 6 to 6 at pH 9 (FIG. 5). Either the protonation state of the enzyme influences the production of 4-HBA versus p-cresol, or the pH modifies the relative stability of the reaction intermediates that lead either to 4-HBA or p-cresol. In addition, as the redox potential of dithionite decreases with increasing pH, this change may also influence the enzymatic production of 4-HBA vs. p-cresol.

In Vivo Production of 4-HBA in a Model Bacterium

E. coli BL21 as detailed above were grown at 37° C. in minimum medium containing NAH₂PO₄ (42 mM); KH₂PO₄ (22 mM); NH₄Cl (19 mM); NaCl (8.5 mM); Thiamine (3 mM); MgSO₄ (2 mM); (NH4 SO₄)₂; CaCl₂ (0.1 mM); Glucose (22 mM) and tyrosine 1 mM.

LC-MS³ detection of 4-HBA was made using a Pepmap100 C₁₈ (Dionex, 100 Å, 15 cm) column at a flow rate of 300 nl·min⁻¹ in 10 mM ammonium acetate. Elution was performed with CH₃CN (0 to 80%). Detection was made on a linear ion trap mass spectrometer (LTQ Standard, Thermo Scientific) in negative mode.

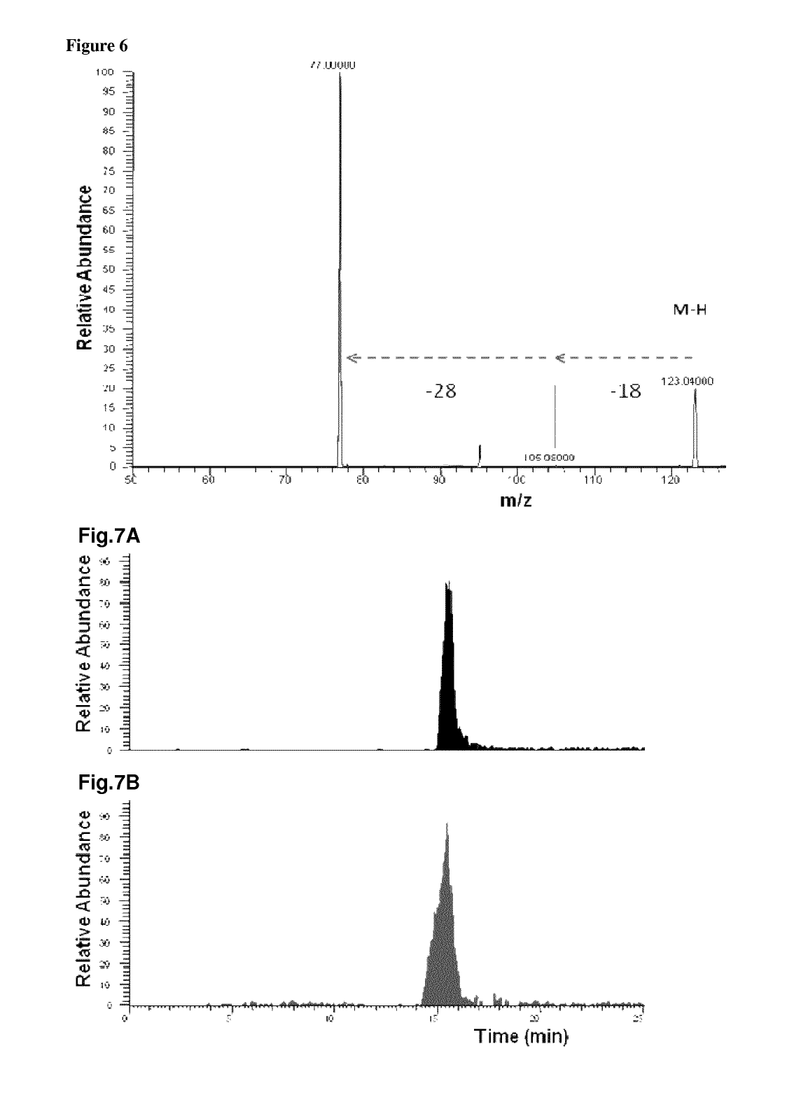

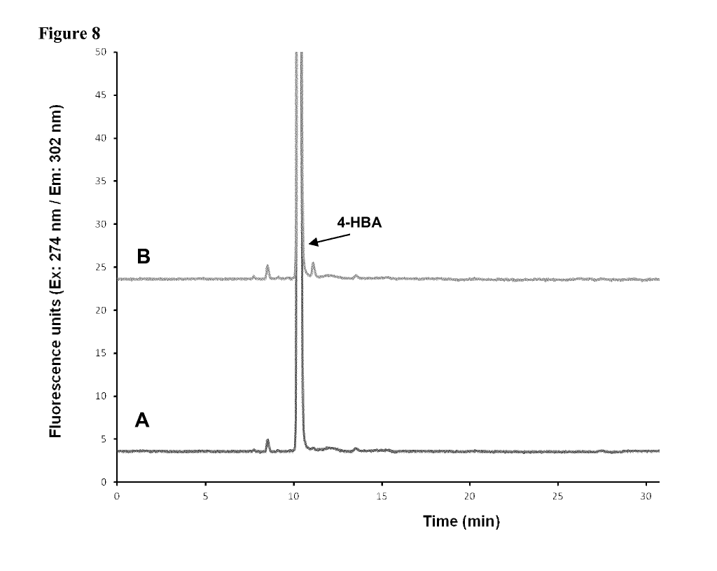

Using LC-MS³ analysis the inventors evidenced 4-HBA in minimal medium when E. coli was grown in the presence of tyrosine and expressing ThiH from Moorella Thermoacetica (FIGS. 6 and 7).

Further experiments were performed using an HPLC system (Agilent, 1290 Infinity) coupled with fluorescence detection (FIG. 8). Tyrosine, 4-HBA and p-cresol were separated using a C18 column (LiChroSpher100, RP-18e, 5 μM, Merck) under a gradient of 0 to 80% CH₃CN containing 0.1% trifluoroacetic acid. Elution was performed at a flow rate of 1 mL·min⁻¹.

E. coli cells expressing either ThiH from Moorella Thermoacetica proved to provide significant amounts of 4-HBA (FIG. 8).

As shown, when the ThiH enzyme from Moorella Thermoacetica was over-expressed in a recombinant host cell, 4-HBA was produced.

TABLE 1

| | CH3 | CH2 | C1 | C2 | C3 | C4 | COOH |
|---|---|---|---|---|---|---|---|
| Tyrosine | 39.0 | 57.0 | 124.4 | 130.2 | 117.5 | 161.9 | 181.7 |
| p-cresol | 19.0 | | 127.3 | 129.8 | 116.1 | 156.6 | |
| In mixture | | | | | | | |
| tyrosine | 36.4 | 57.0 | 127.8 | 131.9 | 116.9 | 155.8 | 175.2 |
| glycine | 42.6 | 173.7 | | | | | |
| | ¹J = | ¹J = | | | | | |
| | 53.8 | 53.9 | | | | | |
| Glyoxylate | 85.1 | 173.3 | | | | | |
| hydrate | ¹J = | ¹J = | | | | | |
| | 55.6 | 55.6 | | | | | |
| 4-hydroxyl | | 64.5 | 133.1 | 130.6 | 116.5 | 156.04 | X |
| benzyl alcohol | | | | | | | |

1-16. (canceled)

17. A method for producing 4-hydroxyl benzyl alcohol (4-HBA) or an analog thereof comprising contacting tyrosine or an analog thereof with an enzyme comprising an amino acid sequence having at least 80% identity with SEQ ID NO: 2 and capable of producing 4-HBA and p-cresol from L-tyrosine, and optionally recovering 4-HBA or the analog thereof.

18. The method of claim 17, wherein the enzyme comprises an amino acid sequence having at least 90% identity with SEQ ID NO: 2.

19. The method of claim 17, wherein the enzyme comprises the amino acid sequence of SEQ ID NO: 2.

20. The method of claim 17, wherein the enzyme is contacted with tyrosine.

21. The method of claim 17, wherein the enzyme is contacted with tyrosine or an analog thereof in the presence of S-adenosyl-L-methionine (SAM).

22. The method of claim 17, wherein the method further comprises purification of 4-HBA or the analog thereof.

23. A recombinant nucleic acid construct or vector comprising a nucleic acid sequence encoding an enzyme comprising an amino acid sequence having at least 80% identity with SEQ ID NO: 2 and capable of producing 4-HBA and p-cresol from L-tyrosine.

24. A recombinant host cell comprising:
a) a recombinant nucleic acid construct or vector comprising a nucleic acid sequence encoding an enzyme comprising an amino acid sequence having at least 80% identity with SEQ ID NO: 2 and capable of producing 4-HBA and p-cresol from L-tyrosine; or
b) a nucleic acid sequence encoding an enzyme comprising an amino acid sequence having at least 80% identity with SEQ ID NO: 2 and capable of producing 4-HBA and p-cresol from L-tyrosine.

25. A method for producing an enzyme capable of producing 4-HBA and p-cresol from L-tyrosine, comprising either cultivating the host cell of claim 24 under conditions conducive for production of the enzyme or expressing the enzyme in vitro, and recovering and/or purifying the enzyme.

26. A solid support on which is immobilized an enzyme comprising an amino acid sequence having at least 80% identity with SEQ ID NO: 2 and capable of producing 4-HBA and p-cresol from L-tyrosine.

27. A composition comprising an enzyme comprising an amino sequence having at least 80% identity with SEQ ID NO: 2 and capable of producing 4-HBA and p-cresol from L-tyrosine, and optionally further comprising S-adenosyl-L-methionine, iron, sulfur, a reducing agent and/or another source of electrons.

28. A method for producing 4-hydroxyl benzyl alcohol (4-HBA) or an analog thereof comprising culturing a host cell of claim 24 in a medium comprising tyrosine or an analog thereof, and optionally recovering the 4-HBA or the analog thereof.

29. A method for producing a compound of interest, comprising producing 4-HBA or an analog thereof by the method according to claim 17 and using the 4-HBA or the analog thereof for producing the compound of interest.

30. The method of claim 29, wherein the compound of interest is selected from the group consisting of p-hydroxybenzaldehyde, p-hydroxybenzoic acid, bisoprolol, 4,4′-dihydroxydiphenylmethane, vanillin and polymers.
